(19) United States
(12) Patent Application Publication
(10) Pub. No.: US 2021/0402006 A1
(43) Pub. Date: Dec. 30, 2021

(54) NANOPARTICLES AND USES THEREOF

(71) Applicant: LIVERPOOL JOHN MOORES UNIVERSITY, Liverpool (GB)

(72) Inventors: Imran SALEEM, Liverpool (GB); Kan KANEKO, Liverpool (GB)

(73) Assignee: LIVERPOOL JOHN MOORES UNIVERSITY, Liverpool (GB)

(21) Appl. No.: 17/293,764

(22) PCT Filed: Nov. 13, 2019
(86) PCT No.: PCT/GB2019/053218

§ 371 Date: May 13, 2021

(30) Foreign Application Priority Data
Nov. 13, 2018 (GB) 1818517.3

(51) Int. Cl. A61K 47/69; A61K 47/61; A61K 9/00; A61K 39/09

(52) U.S. Cl. CPC A61K 47/6937; A61K 47/61; B82Y 5/00; A61K 39/092; A61K 9/0078

(57) ABSTRACT

The present invention relates to a nanoparticle comprising a polymer, α-Galactosylceramide (α-GalCer) and chitosan. The invention also relates to multivalent immunogenic compositions. The invention has particular use as a pulmonary vaccine.

### TECHNICAL FIELD OF THE INVENTION

The present invention relates to nanoparticles and has particular uses as, but not limited to, pulmonary vaccines.

### BACKGROUND TO THE INVENTION

The currently commercially available vaccine for pneumonia (Prevnar 13®, marketed by Pfizer) is composed of cell capsule sugars from Streptococcus pneumonia, which are conjugated to a carrier protein extracted from diptheria. The vaccine has reduced the incidence of pneumococcal disease in England and Wales by more than half and protects against 13 types of bacteria. Despite these statistics, there is geographical variation in serotypes and risks of non-covered strains becoming more prevalent and replacing the strains that can be successfully targeted by the vaccine. Moreover the existing vaccine does not provide mucosal protection through which pathogens enter i.e. nose and lungs. Thus, induction of immunity in the lung mucosa is likely to make an important contribution to protective immunity towards respiratory diseases.

Since the initial discovery of α-GalCer, its immunostimulatory properties have been investigated for a number of applications ranging from cancer to allergies. There have been studies investigating the protective effects of α-GalCer in a Streptococcus pneumoniae infection [1]. These studies found that α-GalCer administration prior to and during infection, resulted in enhanced host defences. These effects were likely due to the activation of natural killer T (NKT) cells and the subsequent production of interferon (IFN-γ) and interleukin-17A (IL-17A), which appear to have a significant influence on the bacterial clearance. However, these immune responses were not due to adaptive or memory immune responses. α-GalCer has has been used successfully in preclinical laboratory formulations against other pathogens such as influenza [2, 3].

The use of proteins (and in particular PspA and derivatives thereof) associated with S. pneumoniae bacteria with nanoparticles would be of interest for vaccine use, as it is recognised as a virulence factor that offers potentially greater coverage between S. pneumoniae serotypes and induces high antibody titres [4]. These immunogenic properties of proteins, such as PspA, are advantageous compared to the capsular polysaccharides, which are T cell independent and induces a poor memory response. Until now, there has been little investigation of PspA in nanoparticulate formulations. There have only been two reported investigations which involved the use of polyanhydride nanoparticles [5] and chitosan-DNA nanoparticles [6], administered through the intranasal route. Both studies showed that the PspA incorporated formulations could elevate the serum levels of anti-PspA IgG antibody in mice. The use of PspA has also been investigated in a nanogel [7] and a live attenuated vaccine [8].

Pneumolysin (and derivatives thereof) is also another virulence factor, which is found in the cytoplasm of all pneumococcal serotypes. It functions as a toxin, binding to cholesterol and subsequently oligomerise to create transmembrane pores. The use of detoxified derivative of pneumolysin (PdT) combined with PspA, has been investigated in the past, showing protective responses in animal models. The administration of the mixture of PspA and PdT to mice, which are subsequently infected with S. pneumoniae, resulted in greater protection to the mice [9]. In addition, the use of hybrid proteins prepared through the genetic fusion of PspA and pneumolysin derivatives showed increased response by cross-reactive antibodies [10].

It is an object of the present invention to overcome or alleviate one of more of the above identified problems. It is also an object of the present invention to provide treatments for a number of pulmonary conditions. It would be preferable if such a treatment could be used to treat or vaccinate against pneumococcal disease or infection.

### SUMMARY OF THE INVENTION

In accordance with a first aspect of the present invention, there is provided a nanoparticle comprising a polymer, α-Galactosylceramide (α-GalCer) and chitosan.

The polymer may comprise a synthetic polymer. Furthermore, the polymer may comprise a number of biodegradable polymers which are suitable for use with medicines or foodstuffs. Preferably, the polymer comprises poly(lactic-co-glycolic acid) (PLGA).

A number of different chitosans may be employed, but it is preferred that the chitosan is water soluble, and most preferably the chitosan is chitosan hydrochloride as chitosan hydrochloride is more soluble in water. The chitosan may comprise chitosan aspartate (ASP) or chitosan-g-acrylamidoglycolic acid (AGA), which are derived from non-animal source.

The nanoparticle will preferably further comprise an antigen. The antigen may be absorbed on, or conjugated to, a surface of the nanoparticle. The antigen may be absorbed on to the surface of the nanoparticle by van der waals or electrostatic interaction. Hence, optimisation can be achieved by varying ratio of antigen to nanoparticle, the concentration of antigen and time of adsorption. The preferred antigen used will of course depend on what immune response is required and which condition or infection the nanoparticle is directed towards addressing. In one embodiment of the invention, the antigen is selected from one or more of the following: pneumococcal surface protein A (PspA) and/or pneumolysin (PdT). In an alternative embodiment of the invention, the antigen is a combination of pneumococcal surface protein A (PspA) and pneumolysin (PdT). The PspA may be recombinant and/or PdT is a detoxified derivative.

In certain embodiments, the nanoparticle is in a dry powder format suitable for inhalation and delivery to the lungs. The dry powder format may be produced by a number of methods, but it has been found that spray drying the nanoparticles is preferred. If desired, the nanoparticles may be combined with one or more excipients. The nanoparticles may be provided in a buffered or saline solution or suitable for mixing with a buffered or saline solution prior to administration. Administration to the lungs may be by traditional dry powder medical inhalers or nebulisers. Depending on administration route, the nanoparticles may be embedded within microcarriers via spraydrying to produce dry powders suitable for inhalation and subsequent delivery to the lungs. Delivery to the lungs may also be effected through the nose or nasal cavity

It is envisaged that advantageously, the nanoparticle system with adsorbed protein for pulmonary mucosal delivery will initiate mucosal, systemic and long-term memory immune response. This will be achieved utilising the PLGA, natural polymer, chitosan, and lipid, a-GalCer to form nanoparticles or nanocarriers and delivered to the lungs as a dry powder or by means of a nebulisation.

Preferably, the nanoparticles are for initiating an immune response. The immune response may comprise a systemic and/or local immune response. Advantageously, it has been found that the nanoparticles of the present invention illicit a systemic and local immune response. For instance, the nanoparticle can induce both systemic and local immune responses for protection against S. pneumoniae.

The nanoparticle may have a number of uses in medicine and research. The nanoparticle may be for use in the delivery and/or presentation of an antigen. The nanoparticle may be for use as a medicament. Preferably, the medicament will be formulated for delivery to and/or through mucosa, and in particular, the lung mucosa.

In certain aspects of the invention, the medicament is a vaccine, and in particular a pulmonary vaccine.

Additionally or alternatively, the medicament may be used as an adjuvant medicament.

In an aspect of the present invention, there is provided a nanoparticle as herein above described, for use in the prevention, management, amelioration or treatment of pneumococcal disease or infection. Such a pneumococcal disease may comprise pneumonia.

In an alternative aspect of the present invention, there is provided a method of prevention, management, amelioration or treatment of a pneumococcal disease or infection, the method comprising administering a therapeutically effective amount of the nanoparticle as herein above described, in an subject in need thereof. Such a pneumococcal disease may comprise pneumonia.

In yet a further alternative aspect of the present invention, there is provided a nanoparticle as herein above described, for use in the manufacture of a medicament for the prevention, management, amelioration or treatment of a pneumococcal disease or infection. Such a pneumococcal disease may comprise pneumonia.

In a further alternative aspect of the present invention, there is provided a nanoparticle as claimed in any preceding claim, for use as an adjuvant.

Advantageously, the combination of a nanocarrier incorporating combinations of antigens (PspA, pneumolysin, etc) and α-GalCer, which is delivered into the lungs, and can induce both systemic and local immune responses for protection against S. pneumoniae. The use of the antigen combinations promotes greater protective responses, mediated through cross-reactive antibodies. The nanocarrier, such as nanoparticles composed of polymers, increases the immunogenicity of the antigens and also promote uptake by the antigen presenting cells such as the dendritic cells. Lastly, the incorporation of α-GalCer should result in a robust immune response which can recruit the T cells lead to appropriate memory cells required for optimal protection.

Typically, prior art vaccines rely on antigens which are generally encapsulated within nanocarriers, and the process of production involves organic solvents and high shear forces, which can denature the antigen. In the present invention, the use of surface absorption of the antigen onto the nanocarrier which occur in aqueous media and the stability of antigen is formed by a protective complex.

The majority of pathogens infect their hosts through mucosal surfaces, such as the lungs. Administration via the parenteral route initiates poor lung mucosal. Thus, induction of immunity in the lung mucosa is likely to make an important contribution to protective immunity towards respiratory diseases or infections.
PLGA, chitosan and α-GalCer have adjuvant and immunostimulatory properties. Hence, the proposed nanocarrier system avoids the use of additional expensive adjuvants, which can also cause inflammatory effects in patients.

This particular combination of the described components has not been previously reported and potentially offers greater protective responses, mediated through cross reactive antibodies. The delivery of these nanoparticles by the lung is also a relatively novel approach and offers benefits associated with non-parenteral administration.

In another aspect of the present invention, there is provided a multivalent immunogenic composition comprising:
a) a nanoparticle comprising a polymer, α-Galactosylceramide (α-GalCer) and chitosan; and
b) a plurality of capsular polysaccharides from Streptococcus pneumoniae serotypes absorbed on, or conjugated to, a surface of the nanoparticle.

Preferably, the capsular polysaccharides are selected from one or more of the Streptococcus pneumoniae serotypes: 1, 3, 4, 5, 6A, 6B, 7F, 9V, 14, 18C, 19A, 19F and 23F.

The composition as herein above described, may be for use as a medicament. Such a medicament may be formulated for delivery to and/or through mucosa, and in particular the lung mucosa.

The medicament may be a vaccine, such as a pulmonary vaccine. Alternatively or additionally, the composition may be used as an adjuvant medicament.

The composition may be for use in the prevention, management, amelioration or treatment of pneumococcal disease, such as pneumonia.

Immunogenic compositions of the invention may, in certain embodiments, further comprises one or more of an adjuvant, a buffer, a cryoprotectant, a salt, a divalent cation, a non-ionic detergent, and an inhibitor of free radical oxidation.

In accordance with another aspect of the present invention, there is provided a method of producing a nanoparticle for the delivery and/or presentation of one or more antigens, the method comprising:

a) forming an oil-in-water emulsion of a polymer and α-Galactosylceramide (α-GalCer) in an organic solvent and agitating the emulsion;

b) mixing the emulsion with an aqueous phase containing chitosan so as to form a mixture and removing the organic solvent from the mixture so as to form a nanoparticle suspension;

c) removing any unbound α-Galactosylceramide (α-GalCer) and/or chitosan from the nanoparticle suspension;

d) absorbing or conjugating the one or more antigens on to the nanoparticles; and optionally separating the nanoparticles which have absorbed or conjugated antigens present.

Preferably, the method further comprises: e) drying the nanoparticles. Step e) may comprise spray drying, and optionally comprises spray drying the nanoparticles together with one or more excipients.

The oil-in water emulsion in a) may further comprise polyvinyl alcohol (PVA), and optionally, the PVA is subsequently removed from the mixture in b).

The agitation in a) may be effected by a number of means which will be apparent to the skilled addressee, but preferably, it is by sonification.

The nanoparticles formed by the method will preferably have a diameter in the range of 100 to 500 nm. More preferably, the nanoparticles formed will have a diameter in the range of 150 to 300 nm. Even more preferably, the nanoparticles formed will have a diameter in the range of 200 to 250 nm. Most preferably, the nanoparticles formed will have a diameter of around 220 nm. The polymer may comprises poly(lactic-co-glycolic acid) (PLGA), the chitosan may comprise chitosan hydrochloride (including ASP or AGA chitosans), and the one or more antigens are absorbed on to the surface of the nanoparticle by van der waals or electrostatic interaction.

Nanoparticles formed with ASP and AGA chitosan will preferably have a diameter in the range of 165 to 175 nm prior to PspA coating. More preferably, the nanoparticles formed with ASP and AGA chitosan will preferably have a diameter in the range of 167 to 172 nm prior to PspA coating. Even more preferably, the nanoparticles formed with ASP and AGA chitosan will preferably have a diameter of around 170 nm prior to PspA coating.

Nanoparticles formed with ASP chitosan will preferably have a diameter in the range of 190 to 210 nm prior to PspA coating. More preferably, the nanoparticles formed with ASP chitosan will preferably have a diameter in the range of 195 to 201 nm prior to PspA coating. Even more preferably, the nanoparticles formed with ASP chitosan will preferably have a diameter of around 197 nm prior to PspA coating.

Nanoparticles formed with AGA chitosan will preferably have a diameter in the range of 220 to 250 nm prior to PspA coating. More preferably, the nanoparticles formed with AGA chitosan will preferably have a diameter in the range of 229 to 237 nm prior to PspA coating. Even more preferably, the nanoparticles formed with AGA chitosan will preferably have a diameter of around 233 nm prior to PspA coating.

The nanoparticles may be embedded within microcarrier formed by spray drying so as to form particles having a diameter in the range of 1 to 5 μm.

In certain aspects, the one or more antigens are selected from proteins obtained from or derived from S. pneumoniae, or protein derivatives thereof. Preferably, the one or more antigens are selected from one or more of the following: pneumococcal surface protein A (PspA) and/or pneumolysin (PdT) and/or derivatives thereof. The PspA may be recombinant and/or PdT is a detoxified derivative.

In an alternative aspect, the one or more antigens may comprise a plurality of capsular polysaccharides from Streptococcus pneumoniae serotypes. Such capsular polysaccharides may be selected from one or more of the Streptococcus pneumoniae serotypes: 1, 3, 4, 5, 6A, 6B, 7F, 9V, 14, 18C, 19A, 19F and 23F.

Preferably, the method as hereinabove described may be used for the production of a nanoparticle as hereinabove described or a multivalent immunogenic composition as hereinabove described.

As used herein, the terms “treatment”, “treating”, “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting or slowing its development; and (c) relieving the disease, i.e., causing regression of the disease.

The term “subject” used herein includes any human or nonhuman animal. The term “nonhuman animal” includes all mammals, such as nonhuman primates, sheep, dogs, cats, cows, horses.

A “therapeutically effective amount” refers to the amount of the nanoparticles that, when administered to a subject for treating a disease, is sufficient to affect such treatment for the disease. The “therapeutically effective amount” will vary depending on active ingredient(s) used, the disease and its severity and the age, weight, etc., of the subject to be treated.

Pharmaceutically acceptable ingredients are well known to those skilled in the art, and include, but are not limited to, pharmaceutically acceptable carriers, adjuvants, excipients, diluents, fillers, buffers, preservatives, 3 carriers, excipients, diluents, fillers, buffers, preservatives, anti-oxidants, lubricants, stabilisers, solubilisers, surfactants (e.g. wetting agents), masking agents, colouring agents, fragrance agents and penetration agents.

Features, integers, characteristics, compounds, molecules, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and figures), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

Embodiments of the invention are described below, by way of example only, with reference to the accompanying figures in which:

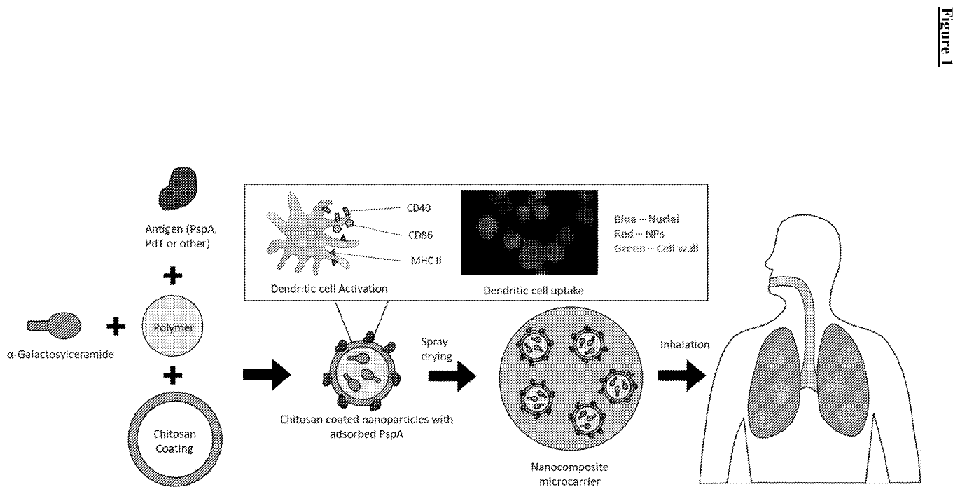

FIG. 1 is a schematic diagram of the nanocarrier system of the present invention and the proposed use as an inhalable vaccine for pneumonia;

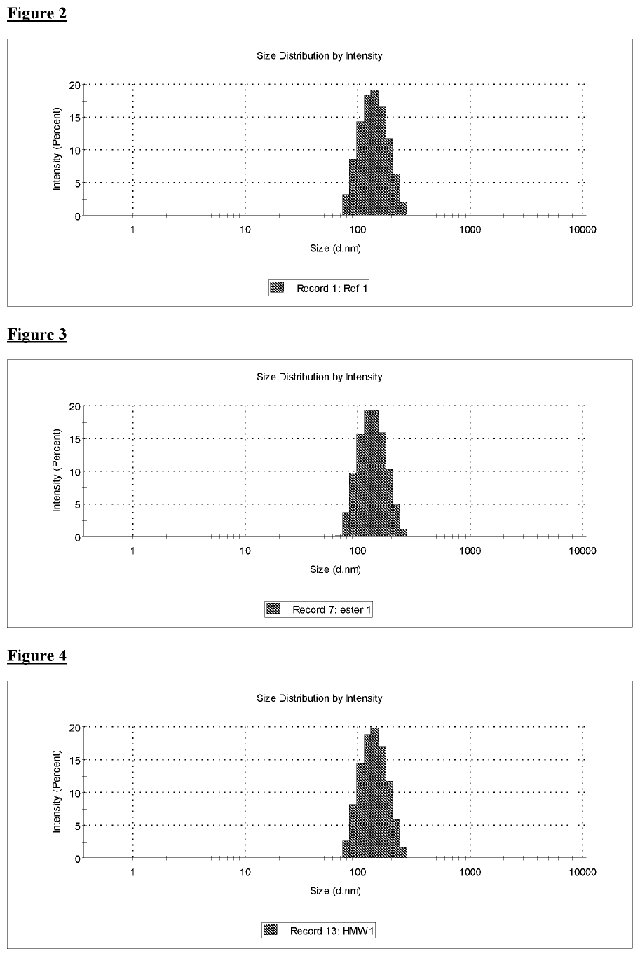

FIG. 2 is a graph showing the size distribution intensity of PLGA (reference) nanoparticles formed without PspA (the data represented is a single run);

FIG. 3 is a graph showing the size distribution intensity of PLGA (ester) nanoparticles formed without PspA (the data represented is a single run);

FIG. 4 is a graph showing the size distribution intensity of PLGA (HMW) nanoparticles formed without PspA(the data represented is a single run);

FIG. 5 is a graph showing the size distribution intensity of PLGA (75:25) nanoparticles formed without PspA (the data represented is a single run);

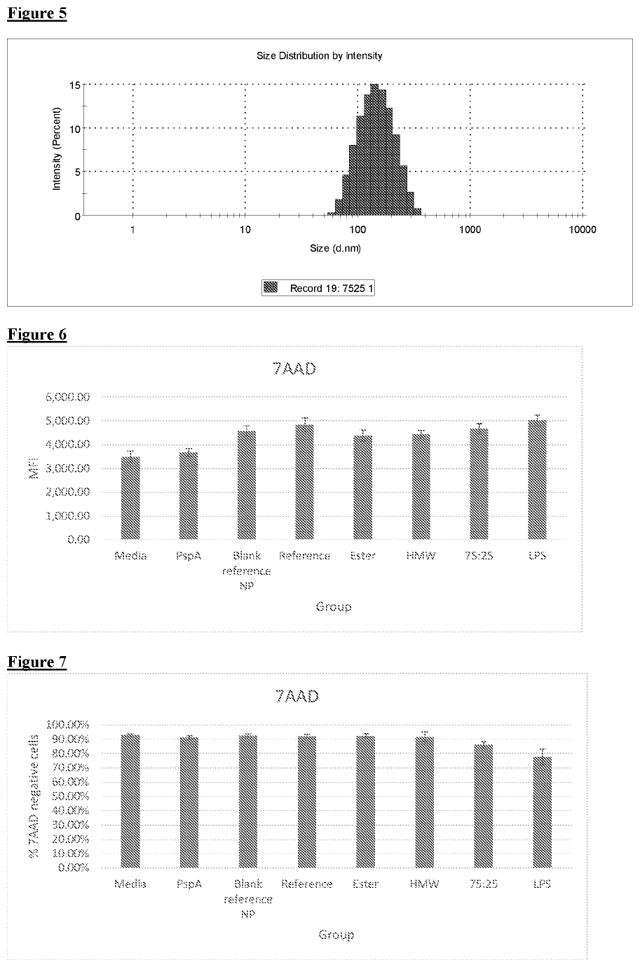

FIG. 6 is a graph showing the median fluorescence intensity (MFI) of 7AAD when the nanoparticles were incubated with JAWS II cells;

FIG. 7 is a graph showing the percentage of 7AAD negative cells when the nanoparticles were incubated with JAWS II cells;

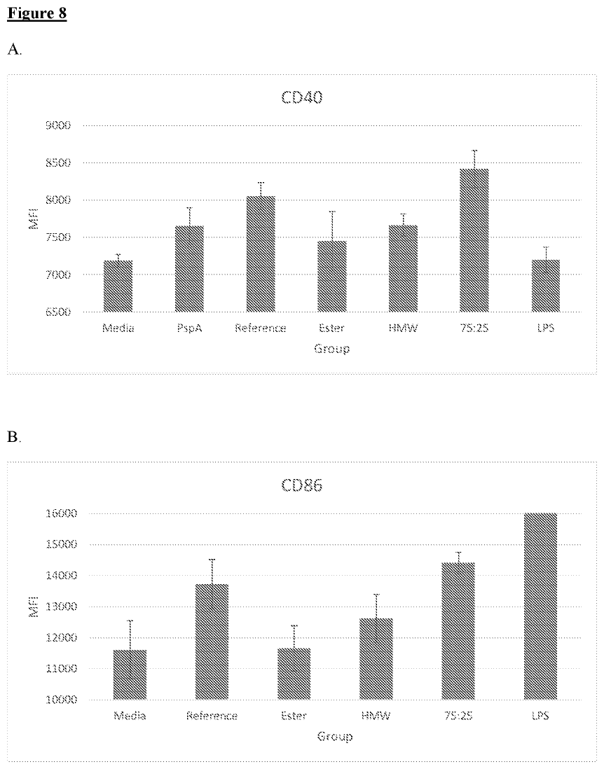

FIG. 8 are graphs showing the median fluorescence intensity (MEI) when the nanoparticles were incubated with JAWS II cells and stained for A. CD40 and B. CD86 respectively;

FIG. 9 is a graph showing the size distribution intensity of PLGA (ref) nanoparticles formed without PspA or chitosan (HCl);

FIG. 10 is a graph showing the size distribution intensity of PLGA (ref) nanoparticles formed with chitosan (HCl) and no PspA;

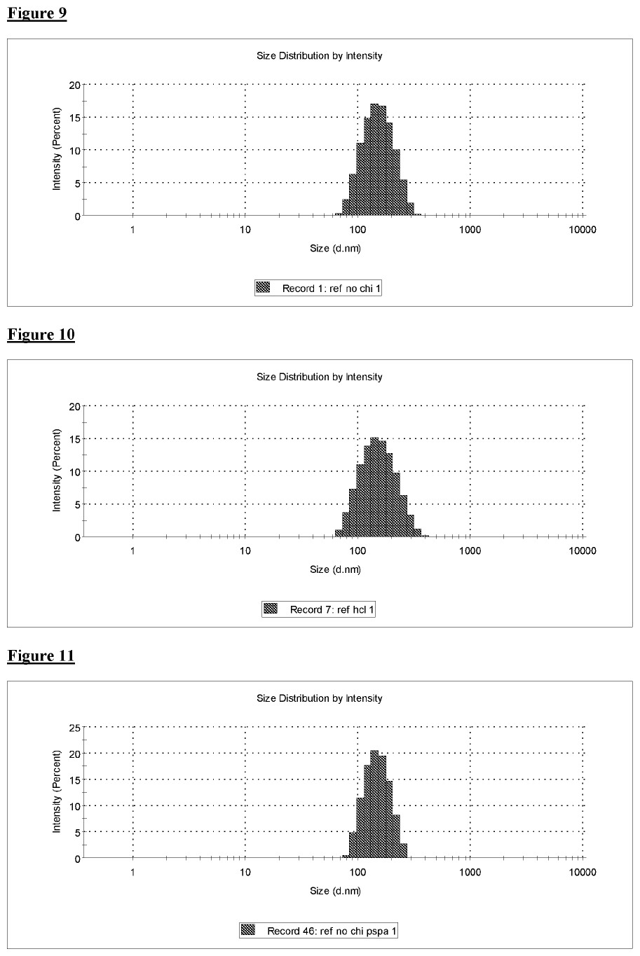

FIG. 11 is a graph showing the size distribution intensity of PLGA (ref) nanoparticles formed with PspA and no chitosan;

FIG. 12 is a graph showing the size distribution intensity of PLGA (ref) nanoparticles formed with chitosan (HCl) and PspA;

FIG. 13 is a graph showing the size distribution intensity of PLGA (75:25) nanoparticles formed with no PspA and no chitosan;

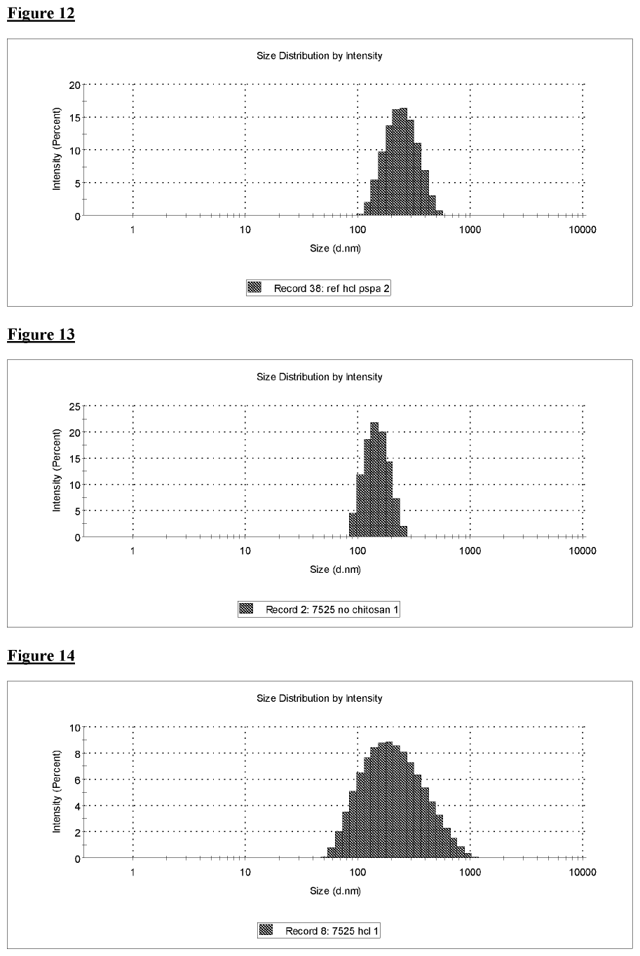

FIG. 14 is a graph showing the size distribution intensity of PLGA (75:25) nanoparticles formed with chitosan (HCl) and no PspA;

FIG. 15 is a graph showing the size distribution intensity of PLGA (75:25) nanoparticles formed with PspA and no chitosan;

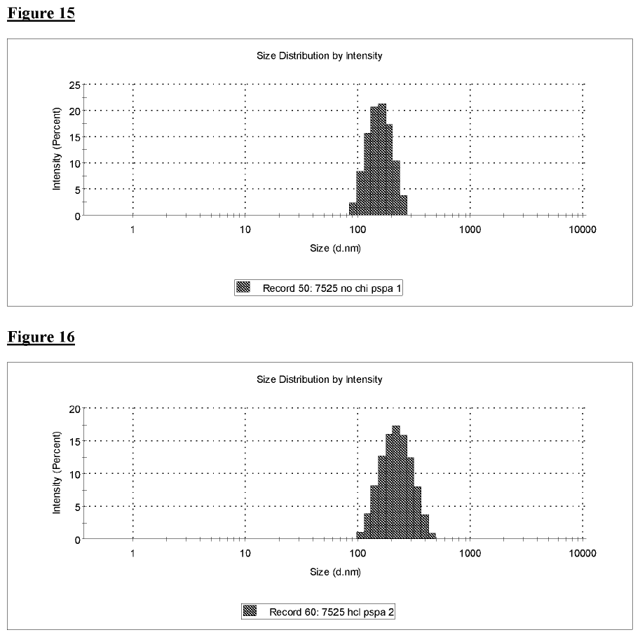

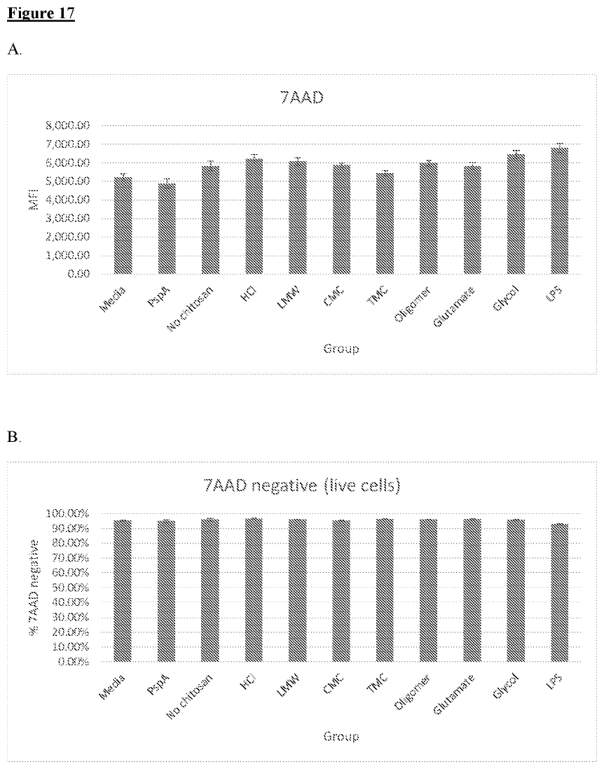

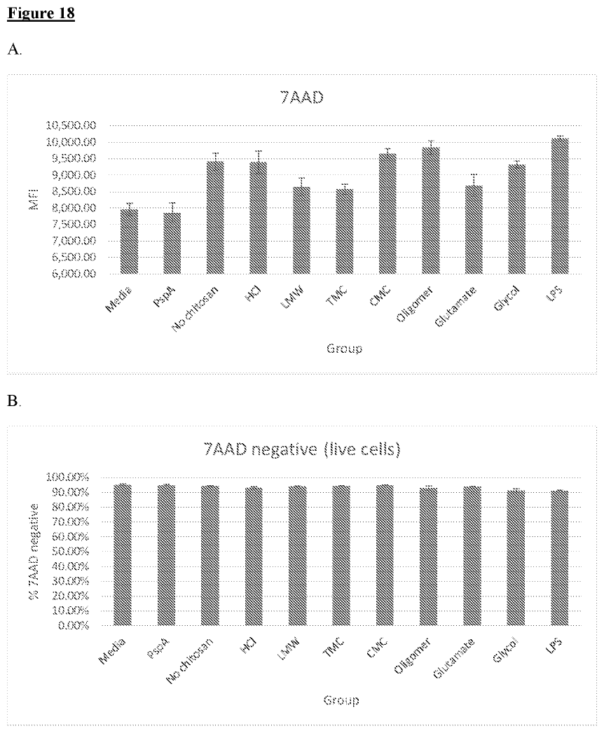

FIG. 16 is a graph showing the size distribution intensity of PLGA (75:25) nanoparticles formed with chitosan (HCl) and PspA;

FIG. 17 are graphs showing the results of when PLGA (ref) nanoparticle formulations with PspA were incubated with JAWS II cells at a concentration of 0.1 mg/mL for 24 hours, and stained with 7AAD to determine the toxicity of the formulations, where A. shows the median fluorescence intensity (MFI) of the 7AAD and B. shows the percentage of 7AAD negative cells;

FIG. 18 are graphs showing the results of when PLGA (75:25) nanoparticle formulations with PspA were incubated with JAWS II cells at a concentration of 0.1 mg/mL for 24 hours, and stained with 7AAD to determine the toxicity of the formulations, where A. shows the median fluorescence intensity (MFI) of the 7AAD and B. shows the percentage of 7AAD negative cells;

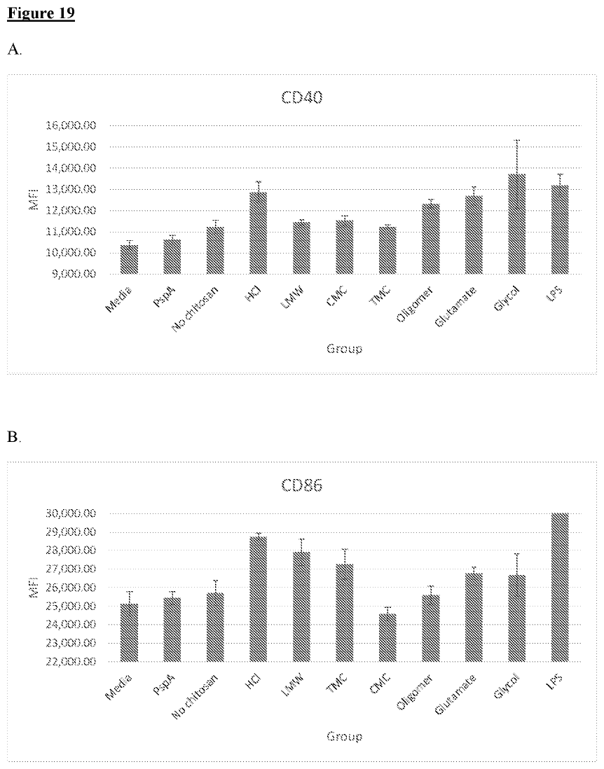

FIG. 19 are graphs showing the median fluorescence intensity (MFI) when Ref+chitosan with PspA nanoparticles were incubated with JAWS II cells and stained for A. CD40 and B. CD86 respectively so as to determine the immunogenicity of each polymer nanoparticle;

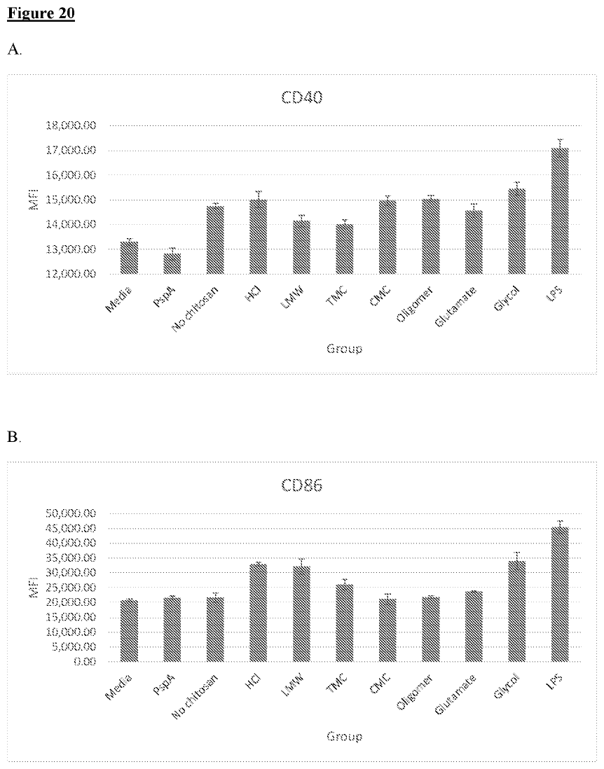

FIG. 20 are graphs showing the median fluorescence intensity (MFI) when 75:25+chitosan with PspA nanoparticles were incubated with JAWS II cells and stained for A. CD40 and B. CD86 respectively so as to determine the immunogenicity of each polymer nanoparticle;

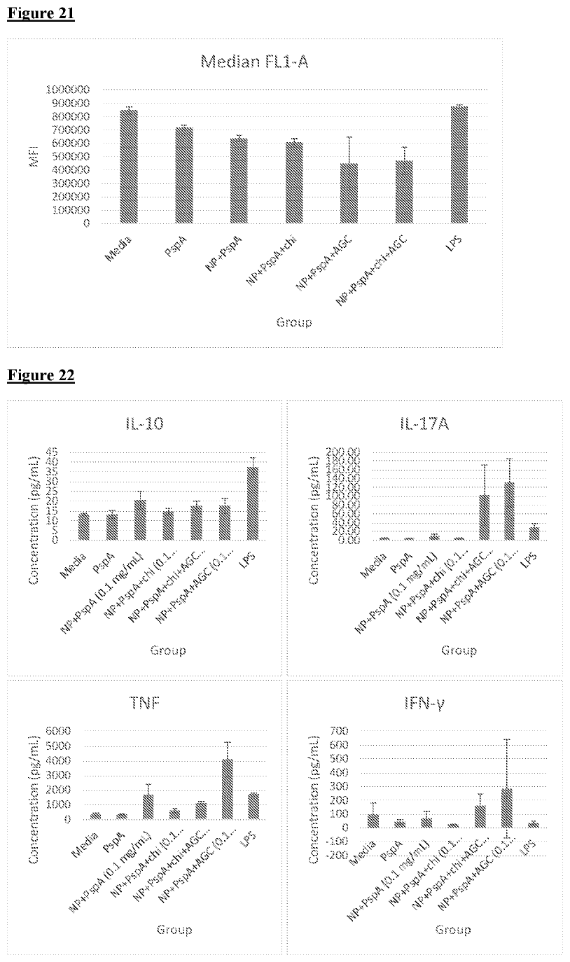

FIG. 21 is a graph showing splenocytes proliferation were splenocytes were stained with CFSE and the median fluorescence intensity (MFI) measured;

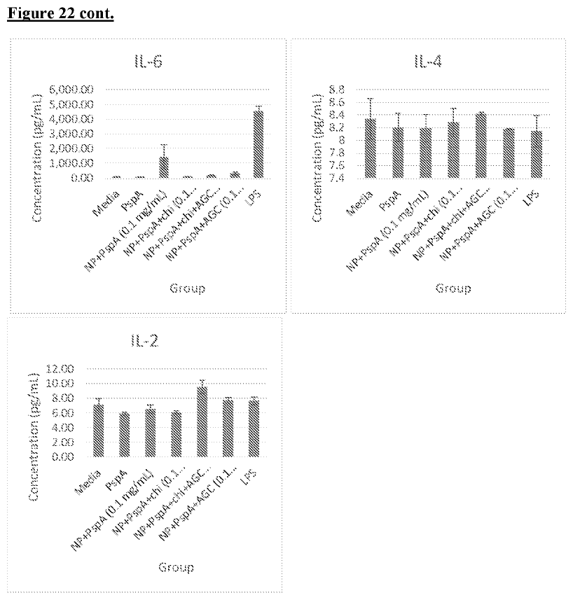

FIG. 22 are graphs showing the cytokine (IL-10, IL-17A, TNF, IFN-gamma, IL-6, IL-4 and IL-2) profiles of the cell culture supernatant from experiments conducted in Example 1;

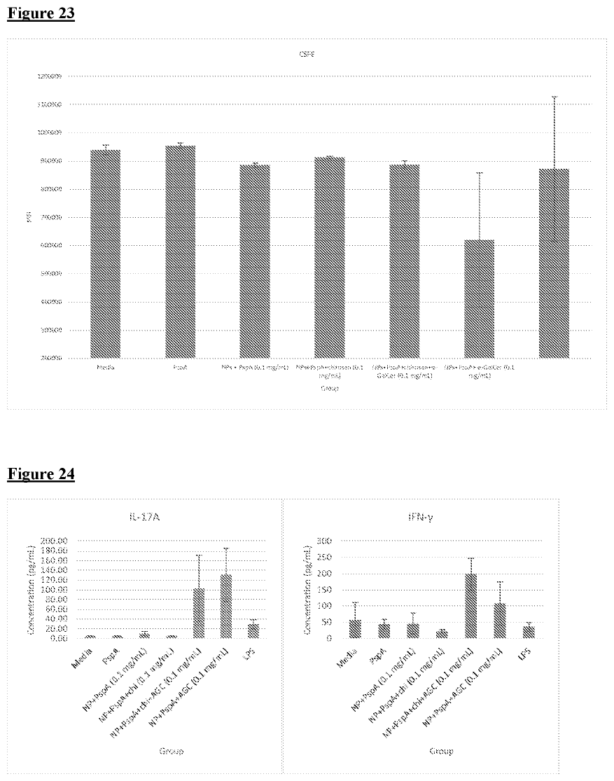

FIG. 23 is a graph showing the median fluorescence intensity (MFI) of various nanoparticles which were incubated with JAWS II cell line and lymphocytes isolated from C57BL/6 mouse spleen to determine the effect of the formulations on the T cells;

FIG. 24 are graphs showing the concentration of cytokine (IL-17A and IFN-gamma) release into the cell culture media after the incubation showing in FIG. 23;

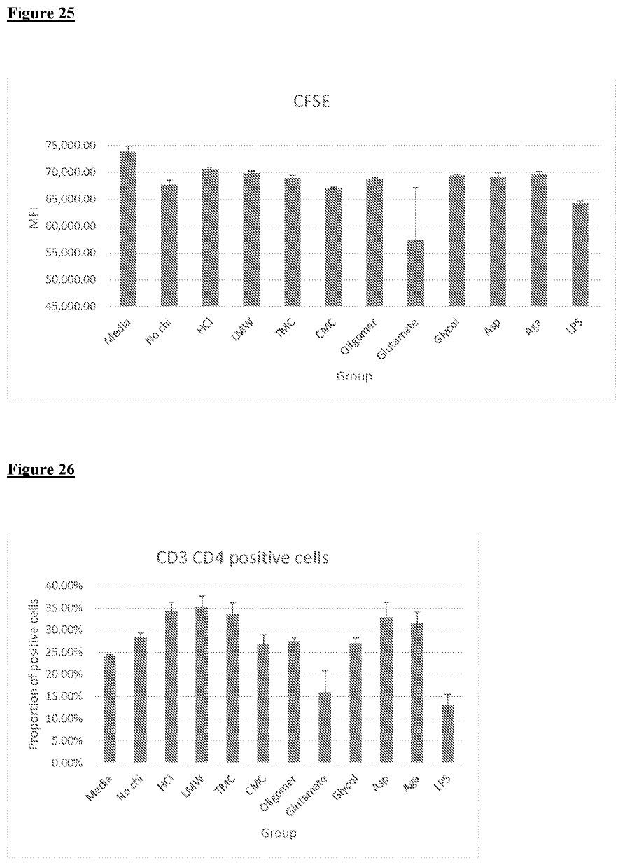

FIG. 25 is a graph showing the CFSE median fluorescence intensity (MFI) after various nanoparticles coated with different chitosans were incubated with the JAWS II cell line and splenocytes, and evaluated for immunogenicity;

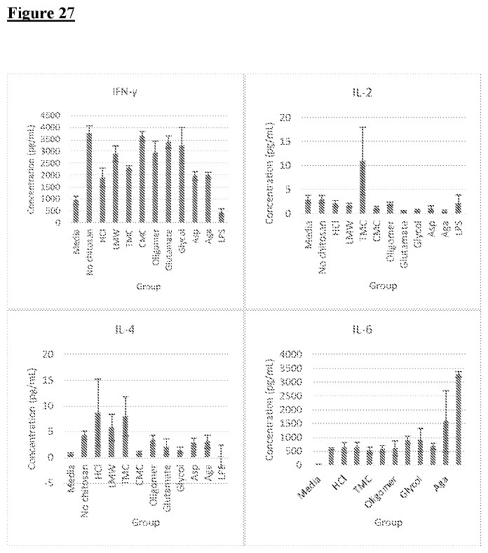

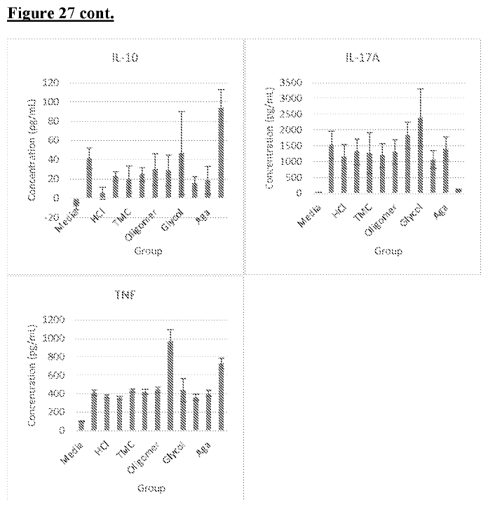

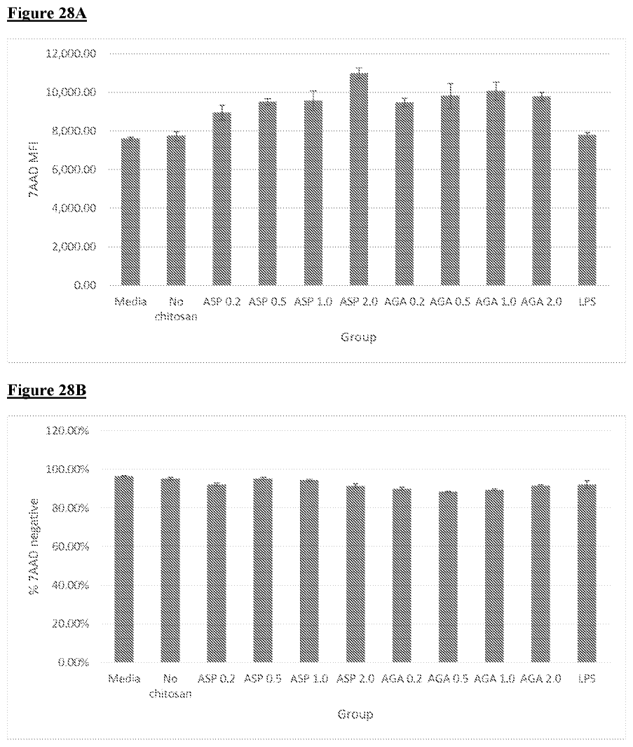

FIG. 26 is a graph showing the proportion of CD3/CD4 positive cells which were measured in the splenocyte population by staining with anti-CD3 and CD4 antibodies and gating for the positive populations;

FIG. 27 are graphs showing the cytokine (IL-10, IL-17A, TNF, IFN-gamma, IL-6, IL-4 and IL-2) profiles of the cell culture supernatant from the experiments conducted in Example 2;

FIG. 28A is a graph showing the median fluorescence intensity (MFI) of 7AAD in JAWSII cells after incubation (24 h) with ASP and AGA chitosan coated PLGA NPs at different concentrations during NP preparation (0.2, 0.5 1.0, 2.0 mg/mL) and FIG. 28B is a graph showing the percentage proportion of 7AAD negative JAWSII cells after incubation (24 h) with ASP and AGA chitosan coated PLGA NPs at different concentrations during NP preparation (0.2, 0.5 1.0, 2.0 mg/mL);

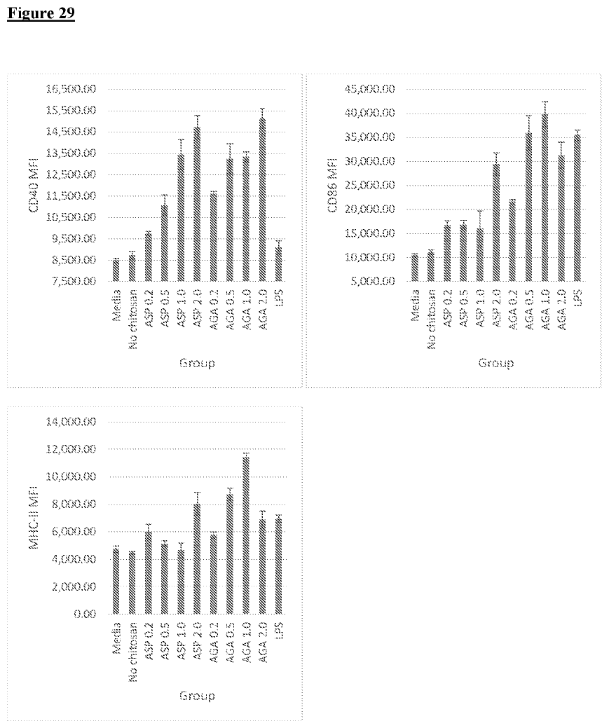

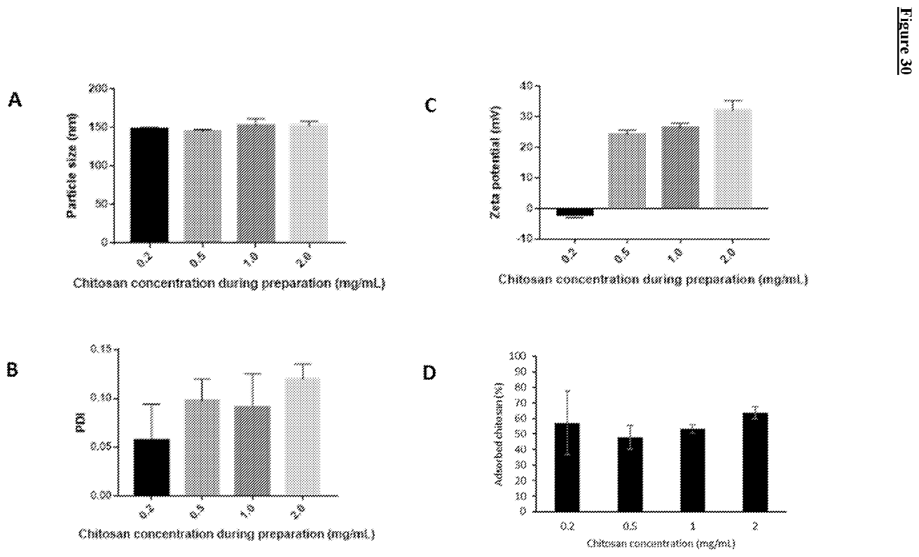

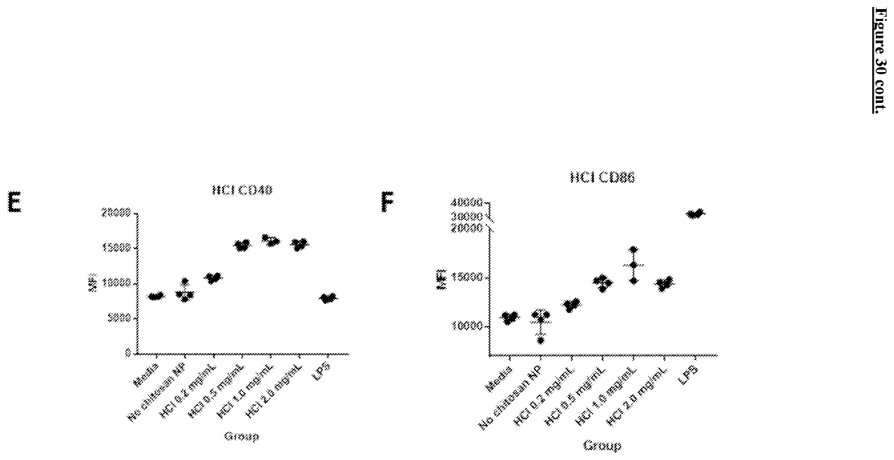

FIG. 29 shows graphs of median fluorescence intensity (MFI) of various nanoparticles which were incubated with JAWS II cell line and shows the activation of the JAWSII cells with CD40, CD86 and MHC-II;

FIGS. 30A-C show chitosan coated PLGA NP characteristics after coating with different chitosan concentrations during preparation (n=3 independent batches), FIG. 30D shows chitosan adsorption at different concentrations during preparation (n=3 independent batches), and FIGS. 30E-F show CD40 and CD86 expression after incubation of chitosan coated PLGA NPs with varying concentrations of chitosan during preparation (0.2, 0.5, 1.0 and 2.0 mg/mL); and

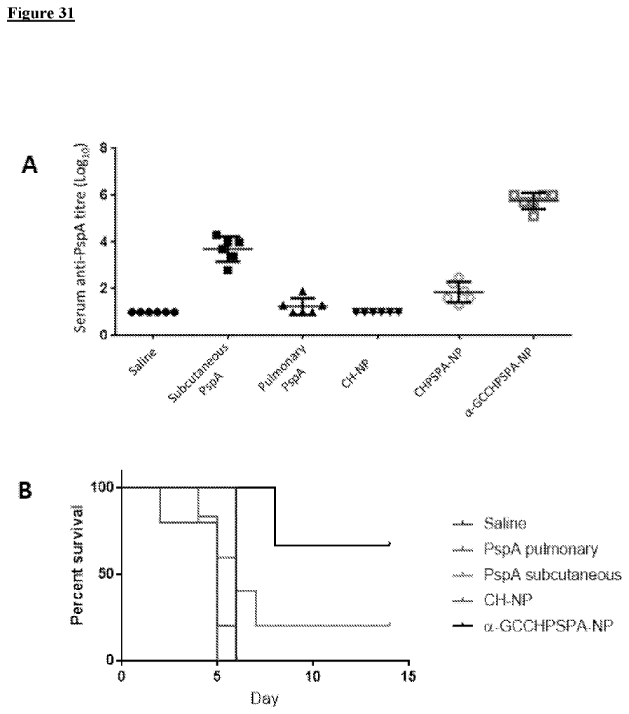

FIG. 31A shows serum anti-PspA titres after administrations of the formulation in BALB/c mice, all formulations were administered into the lungs through the nose (except for the subcutaneous PspA) on day 0 and 14, and the serum was obtained on day 28 (n=6), and FIG. 31B shows survival after challenge with S. pneumoniae (ATC6303), BALB/c mice were immunised with the formulations through the nose, on day −35 and −21, then challenged with the S. pneumoniae on day 1, the mice were monitored for 14 days after challenge (n=6).

The following Examples show the production of a nanocarrier system composed of polymers, made using the solvent evaporation method, and subsequently coated with water-soluble chitosan (as most literature references use non-water soluble chitosan which is dissolved in acetic acid). The selection of polymers such as poly(lactic-co-glycolic acid) (PLGA).

The antigens (PspA, PdT) can be incorporated in the particles, or adsorbed or conjugated to the particle surface. The α-GalCer is incorporated in the layer of the polymer during fabrication of the nanoparticles. The resulting particles are in the nano-size range with polydispersity indexes indicating size range homogeneity.

The process for the preparation of nanoparticles using the solvent evaporation method involves forming an o/w emulsion of the organic solvent containing the polymer and a-GalCer, and water containing polyvinyl alcohol (PVA), using a probe sonicator. The emulsion is then placed into a larger volume of aqueous phase containing the chitosan, and left stirring to allow the organic phase to evaporate. The resulting suspension of polymeric NPs is centrifuged to separate the PVA and unincorporated α-GalCer and chitosan, from the formed NPs. Antigens for surface adsorption are then added. The fabrication of these NPs could be translated to microfluidic methods for upscaled manufacturing.

The resulting nanoparticles can be formulated as microparticles for optimal lung deposition. This is achieved through spray drying of the nanoparticles together with excipients (sugars, amino-acids, buffer salts etc) to obtain nanocomposite microparticles (NCMPs), nanoparticles embedded within microcarriers, of desired size (1-5 μm) and homogeneity for optimum delivery to site with in the lungs for targeting antigen presenting cells, such as dendritic cells. The NCMPs will be formulated with a particle size 1-5 μm and aerosolised into the lung to allow deposition in the respirable airways and dissolve releasing the nanocarrier, as they are produced from water-soluble amino acids and sugars. The nanocarrier will be up taken by DCs within the respirable airways. As a dry powder the cold-chain requirement are eliminated, and the nanocarrier and antigen are more stable. In addition, as a dry powder NCMPs it is much easier to deliver via inhalation by patients and can be used as a single dose treatment. The NCMPs can be dispersed in a nebuliser, and aerosolised into the lungs as above also as a single dose treatment.

### Example 1

### PLGA Polymer Selection

The polymers outlined below in Table 1 were initially selected for investigating the effects of differences in PLGA polymers. These were based on whether the polymer end was an acid or ester, molecular size, and the ratio of lactic acid to glycolic acid, all relative to the reference polymer.

TABLE 1

| | Lactic | | | | Aldrich |
|---|---|---|---|---|---|
| | acid:Gly- | Acid or | Molecular | | product |
| Polymer name | colic acid | ester end | size | | # |
| Reference (Ref) | 50:50 | Acid | 7-17 | kDa | 719897 |
| Ester | 50:50 | Ester | 7-17 | kDa | 719889 |
| High molecular | 50:50 | Acid | 24-38 | kDa | 719870 |
| weight (HMW) | | | | | |
| 75:25 | 75:25 | Acid | 4-15 | kDa | 719919 |

### Particle Characteristics for Different PLGAs:

Nanoparticles (NPs) prepared from the different polymers had sizes that were generally similar. As shown in Table 2 below, the zeta potential of the ester and 75:25 polymer NPs appeared to be lower than those of the reference and HMW polymers. The magnitude of the zeta potential decreased for the reference and ester polymers, but increased for the HMW and 75:25 polymers after coating with PspA.

TABLE 2
(data represents average of n = 3)

| | | | | | Zeta | |
|---|---|---|---|---|---|---|
| | Size | | | | potential | |
| | (nm) | SD | PdI | SD | (mV) | SD |
| Before PspA | | | | | | |
| coating | | | | | | |
| Ref | 157.8 | 1.6 | 0.122 | 0.007 | −29.1 | 0.8 |
| Ester | 151.5 | 8.9 | 0.083 | 0.024 | −17.1 | 1.1 |
| HMW | 151.6 | 1.8 | 0.068 | 0.004 | −16.7 | 0.7 |
| 75:25 | 165.4 | 1.4 | 0.072 | 0.008 | −15.1 | 0.3 |
| After PspA | | | | | | |
| coating | | | | | | |
| Ref | 169.2 | 2.7 | 0.148 | 0.010 | −22.5 | 1.1 |
| Ester | 153.2 | 9.4 | 0.080 | 0.066 | −14.0 | 1.2 |
| HMW | 153.5 | 2.5 | 0.084 | 0.018 | −25.8 | 1.9 |
| 75:25 | 166.4 | 2.5 | 0.118 | 0.003 | −17.4 | 0.3 |

### Particle Size Distribution

The size distribution curves as shown in FIGS. 2 to 5 (and the data also shown in Tables 3 to 6 below) demonstrate that the formed NPs are relatively homogenous in size. The data represented is a single run.

The data for NPs formed without PspA is shown below in Table 3.

TABLE 3

| Average size (nm) | Peak size (nm) | SD (nm) |
|---|---|---|
| 133.0 | 143.3 | 39.91 |

The data for NPs using Ester is shown below in Table 4.

TABLE 4

| Average size (nm) | Peak size (nm) | SD (nm) |
|---|---|---|
| 129.2 | 138.6 | 37.84 |

The data for NPs formed with HMW is shown below in Table 5.

TABLE 5

| Average size (nm) | Peak size (nm) | SD (nm) |
|---|---|---|
| 133.7 | 143.2 | 38.64 |

The data for NPs formed with a ratio of 75:25 is shown below in Table 6.

TABLE 6

| Average size (nm) | Peak size (nm) | SD (nm) |
|---|---|---|
| 136.4 | 154.9 | 55.87 |

### Toxicity

The NP formulations with PspA were incubated with JAWS II cells at a concentration of 0.1 mg/mL for 24 hours, and stained with 7AAD to determine the toxicity of the formulations. The median fluorescence intensity (MEI) of the 7AAD indicates the state of disruption of the cell membrane. The results show that all of the formulations had a noticeable increase in the MFI, although there were no differences between the polymers and also compared to the LPS (positive control for immunostimulation).

As illustrated in FIGS. 6 and 7, when presented as a percentage of viable cells, the formulations maintained a viability of over 80% compared to the media group (negative control).

Surface Marker Upregulation on JAWS II Cells after Incubation for 24 Hours

The NPs formulations with PspA were incubated with JAWS II cells as above, and the cells were subsequently stained for cell surface markers (CD40, CD86 and MHC-II) to determine the immunogenicity of each polymer NP. The groups were; Media only (negative control), PspA only, Reference, Ester, HMW, 75:25, lipopolysaccharide (LPS, positive control). As illustrated in FIG. 8, the Ref and 75:25 generally exhibited the highest surface marker upregulation compared to the HMW and Ester polymers.

### Chitosan Selection

The subsequent goal was to investigate the effect of the type of coated chitosan on the NP formulations. Various chitosans (as detailed in Table 7 below) with different molecular structures were tested to determine their effect on particle properties and immunogenicity.

TABLE 7

| | Molecular | | Degree of | |
|---|---|---|---|---|
| Chitosan name | weight | | deacetylation | Product # |
| HCl | | | 80-95% | 43001 HMC |
| Low molecular weight | 50,000-190,000 | | 75-85% | 448869 Aldrich |
| (LMW) | Da | | | |
| Trimethylchitosan | NA | | NA | Made in lab |
| (TMC) | | | | from LMW |
| Carboxymethylchitosan | | | 80-95% | 43002 HMC |
| (CMC) | | | | |
| Oligomer | <5 | kDa | ≥75% | 44009 HMC |
| Glutamate | NA | | NA | 44006 HMC |
| Glycol | NA | | NA | G7753 Sigma |
| chitosan aspartate | ~200 | kDa | NA | ChiPro |
| (ASP) | | | | |
| chitosan-g- | ~200 | kDa | NA | ChiPro |
| acrylamidoglycolic acid | | | | |
| (AGA) | | | | |

### Particle Characteristics of Different Chitosans

The chitosan coating changed the NP size. As shown in Tables 8 and 9 below, the CMC and glycol chitosans had the greatest effect, increasing the NP size from approximately 150 nm to >200 nm (without PspA) and 140 nm to >300 nm (with PspA). The NP surface charge also differed greatly between the chitosans, with CMC and oligomer chitosans exhibiting negative zeta potential.

### Reference Polymer:

TABLE 8

| | | | | | Zeta | |
|---|---|---|---|---|---|---|
| | Size | | | | potential | |
| | (nm) | SD | PdI | SD | (mV) | SD |
| Before PspA | | | | | | |
| coating | | | | | | |
| No chitosan | 152.7 | 22.9 | 0.121 | 0.083 | −23.1 | 5.1 |
| HCl | 141.6 | 8.8 | 0.146 | 0.032 | 19.6 | 2.2 |
| LMW | 137.9 | 4.8 | 0.124 | 0.008 | 26.9 | 4.7 |
| TMC | 160.9 | 1.2 | 0.185 | 0.036 | 7.8 | 2.9 |
| CMC | 245.1 | 39.1 | 0.226 | 0.040 | −20.6 | 22.9 |
| Oligomer | 167.7 | 8.0 | 0.231 | 0.071 | −6.1 | 1.4 |
| Glutamate | 147.2 | 9.2 | 0.175 | 0.057 | 17.9 | 14.0 |
| Glycol | 195.1 | 44.9 | 0.140 | 0.020 | 27.2 | 14.3 |
| After PspA | | | | | | |
| coating | | | | | | |
| No chitosan | 137.9 | 4.5 | 0.094 | 0.049 | −23.5 | 2.5 |
| HCl | 188.1 | 11.3 | 0.338 | 0.073 | 12.6 | 3.8 |
| LMW | 142.8 | 7.7 | 0.135 | 0.015 | 17.8 | 0.8 |
| TMC | 214.8 | 65.6 | 0.237 | 0.051 | 0.5 | 1.6 |
| CMC | 324.1 | 77.8 | 0.210 | 0.078 | −32.1 | 0.6 |
| Oligomer | 181.8 | 24.9 | 0.251 | 0.103 | −8.5 | 0.9 |
| Glutamate | 164.1 | 16.8 | 0.191 | 0.047 | 23.1 | 1.0 |
| Glycol | 551.4 | 91.0 | 0.401 | 0.040 | 30.3 | 0.9 |

### 75:25 Polymer:

TABLE 9

| | | | | | Zeta | |
|---|---|---|---|---|---|---|
| | Size | | | | potential | |
| | (nm) | SD | PdI | SD | (mV) | SD |
| Before PspA | | | | | | |
| coating | | | | | | |
| No chitosan | 143.1 | 1.7 | 0.071 | 0.032 | −26.9 | 6.7 |
| HCl | 155.7 | 13.2 | 0.164 | 0.067 | 17.6 | 4.1 |
| LMW | 153.0 | 7.5 | 0.152 | 0.012 | 28.3 | 4.3 |
| TMC | 150.0 | 4.8 | 0.090 | 0.018 | 9.1 | 6.6 |
| CMC | 254.1 | 80.3 | 0.194 | 0.063 | −30.6 | 2.6 |
| Oligomer | 161.6 | 12.3 | 0.158 | 0.065 | −5.2 | 0.8 |
| Glutamate | 155.1 | 15.3 | 0.084 | 0.022 | 25.6 | 1.6 |
| Glycol | 287.0 | 30.2 | 0.099 | 0.049 | 33.5 | 1.2 |
| After PspA | | | | | | |
| coating | | | | | | |
| No chitosan | 162.3 | 24.8 | 0.130 | 0.097 | −22.4 | 3.2 |
| HCl | 212.4 | 6.0 | 0.294 | 0.095 | 10.3 | 13.9 |
| LMW | 161.9 | 4.7 | 0.151 | 0.013 | 20.8 | 1.9 |
| TMC | 189.7 | 12.9 | 0.194 | 0.067 | 0.0 | 1.4 |
| CMC | 279.8 | 19.8 | 0.174 | 0.060 | −32.5 | 0.8 |
| Oligomer | 161.3 | 3.9 | 0.152 | 0.028 | −8.6 | 0.6 |
| Glutamate | 270.3 | 12.2 | 0.235 | 0.056 | 17.8 | 3.6 |
| Glycol | 622.4 | 74.7 | 0.245 | 0.089 | 27.8 | 1.0 |

There were some differences between the Ref and 75:25 polymers for each chitosan tested, and the Ref chitosan formulations look to be generally smaller. But the overall pattern appeared similar, with the CMC and glycol chitosans exhibiting the largest particle sizes.

### Particle Size Distribution

The size distribution curves as shown in FIGS. 9 to 16 (and the data also shown in Tables 10 to 17 below) demonstrate that the formed NPs. The data represented is a single run.

Reference PLGA (without PspA):

The data for NPs formed with no chitosan is show below in Table 10.

TABLE 10

| Average size (nm) | Peak size (nm) | SD (nm) |
|---|---|---|
| 142.1 | 157.8 | 49.4 |

The data for NPs formed with HCl is shown below in Table 11.

TABLE 11

| Average size (nm) | Peak size (nm) | SD (nm) |
|---|---|---|
| 145.1 | 160.3 | 58.11 |

Reference PLGA (with PspA):

The data for NPs formed without chitosan is shown in below Table 12.

TABLE 12

| Average size (nm) | Peak size (nm) | SD (nm) |
|---|---|---|
| 142.7 | 152.6 | 39.25 |

The data for NPs formed with HCl is shown below in Table 13.

TABLE 13

| Average size (nm) | Peak size (nm) | SD (nm) |
|---|---|---|
| 235.8 | 255.7 | 83.16 |

75:25 PLGA (without PspA):

The data for NPs formed without chitosan is shown in below Table 14.

TABLE 14

| Average size (nm) | Peak size (nm) | SD (nm) |
|---|---|---|
| 140.3 | 156.1 | 53.67 |

The data for NPs formed with HCl is shown below in Table 15.

TABLE 15

| Average size (nm) | Peak size (nm) | SD (nm) |
|---|---|---|
| 176.3 | 246.2 | 160.6 |

75:25 PLGA (with PspA):

The data for NPs formed without chitosan is shown in below Table 16.

TABLE 16

| Average size (nm) | Peak size (nm) | SD (nm) |
|---|---|---|
| 152.4 | 160.1 | 39.22 |

The data for NPs formed with HCl is shown below in Table 17.

TABLE 17

| Average size (nm) | Peak size (nm) | SD (nm) |
|---|---|---|
| 210.1 | 230.2 | 72.02 |

### Toxicity

The toxicity of Ref+chitosan with PspA & 75:25+chitosan with PspA were evaluated again using 7AAD. The toxicity reflects the pattern shown by the immunogenicity of the Ref chitosan formulations, with the HCl and glycol formulations exhibiting the highest MFI. As shown in FIGS. 17 and 18, the patterns are slightly different between Ref and 75:25, with CMC and oligomer formulations exhibiting the highest MFI for 75:25.

Surface Marker Upregulation on JAWS II Cells after Incubation for 24 Hours

The CD40 and CD86 surface marker upregulation was investigated again in JAWS II cells using Ref+chitosan with PspA & 75:25+chitosan with PspA. As shown in FIGS. 19 and 20, the ref and 75:25 polymers exhibited similar patterns of upregulation, with the HCl and glycol having the highest MEI for CD40, and HCl and LMW having the highest CD86 expression.

From the data obtained, the ref and 75:25 PLGA polymers, and the HCl chitosan induced the highest immunogenicity.

### Co-Culture Model of JAWS II Cells and Mouse Splenocytes

The effect of the 75:25 PLGA HCl chitosan NPs on the T cells was investigated in a co-culture assay, as T cells interact with the dendritic cells (JAWS II cells) and subsequently participates in the immune response. The T cell effect offers insight into the direction of how the immune response will progress. Mouse splenocytes which contain a relatively abundance number of T cells, were incubated with the JAWS II cells and the 75:25 PLGA HCl chitosan NPs, and the splenocyte proliferation and cytokine release were measured after 5 days. The results indicated that the chitosan and α-Galactosylceramide (α-Galcer) both had effect on the immune response by the T cells.

### Splenocyte Proliferation

The splenocytes were stained with CFSE and the MFI was measured to determine degree of cell proliferation. As shown in FIG. 21, the NP with the chitosan and α-GalCer (AGC) exhibited the lowest MFI, indicating greatest degree of cell division.

### Supernatant Cytokine

The cell culture supernatant was obtained and cytokines were measured using cytometric bead array (CBA) assay. As shown in FIG. 22, the NP with PspA and α-GalCer generally exhibited the highest cytokine release. The NP formulations and LPS (positive control) appeared to exhibit different degrees of cytokine release. These experiments were then repeated with ref and 75:25 PLGA with all chitosans and α-GalCer as detailed in Example 2 below.

### Example 2

### Co-Culture Experiment 1

NPs were incubated with DCs (JAWS II cell line) and lymphocytes isolated from C57BL/6 mouse spleen to determine the effect of the formulations on the T cells. As shown in FIG. 23, the NPs with PspA and α-GalCer exhibited the lowest CFSE MFI, indicating proliferation of cells isolated from the spleen.

Cytokine release into the cell culture media was also measured from the above experiment. As shown in FIG. 24, the results indicated that the α-GalCer containing formulations were causing the release of IL-17A and IFN-γ, which may be important as part of the immune response for vaccines.

### Co-Culture Experiment 2

NPs (made from 75:25 PLGA) coated with different chitosans were incubated with the DCs and splenocytes, as in the previous experiment, and evaluated for immunogenicity. As shown in FIG. 25, the lower CFSE MFI signal of the splenocytes indicated proliferation, but there may all chitosan groups exhibited similar results.

The proportion of CD3/CD4 positive cells were measured in the splenocyte population by staining with anti-CD3 and CD4 antibodies and gating for the positive populations. As shown in FIG. 26, the data showed greater CD3/CD4 positive proportions in the HCL, LMW and TMC chitosan groups, indicating T cell proliferation. Lipopolysaccharide (LPS) was unfortunately not the best positive control for the experiment.

As shown in FIG. 27, cytokine release into the cell culture medium was also measured but did not show any consistent pattern.

### Example 3

### Particle Characteristics

Non-animal source ASP and AGA chitosan coated PLGA NPs were assessed and were shown to exhibit similar particle sizes of approximately 170 nm before PspA coating. The particle surface charge differed significantly with the ASP chitosan coated NPs having a +30 mV zeta potential compared to +17 mV for the AGA chitosan. As shown in FIG. 18 below, after coating, the zeta potentials were similar at the low +20 s, whereas the particle size was now different, with the AGA having a greater size increase compared to the ASP chitosan.

TABLE 18

| | Size | | Zeta potential |
|---|---|---|---|
| | (nm) | PdI | (mV) |
| Before PspA adsorption | | | |
| ASP | 171.1 ± 1.9 | 0.151 ± 0.002 | +30.2 ± 1.0 |
| AGA | 169.0 ± 0.1 | 0.101 ± 0.013 | +17.1 ± 0.5 |
| After PspA adsorption | | | |
| ASP | 197.2 ± 2.8 | 0.119 ± 0.033 | +23.1 ± 1.0 |
| AGA | 233.0 ± 4.1 | 0.277 ± 0.041 | +20.2 ± 1.0 |

### PspA and Chitosan Absorption

As shown in Table 19 below, both ASP and AGA chitosan coated NPs exhibit high adsorption of PspA, but differ greatly in adsorption of the respective chitosans.

TABLE 19

| | PspA adsorption efficiency | Chitosan adsorption efficiency |
|---|---|---|
| | (%) | (%) |
| ASP | 90.6 ± 8.1 | 18.2 ± 4.9 |
| AGA | 88.2 ± 11.4 | 50.8 ± 2.1 |

### Toxicity

As shown in FIGS. 28A and 28B, the toxicity of the ASP and AGA chitosan coated PLGA NPs were evaluated in JAWS II cells, which indicated high cell viability of over 88%, compared to the untreated cells which had 96% viability. The 7AAD MFI suggested a chitosan concentration dependent pattern, with highest ASP concentration exhibiting the highest 7AAD MFI.

### DC Activation

As shown in FIG. 29, The activation of the JAWSII cells suggested a concentration dependent pattern for CD40, with higher ASP and AGA chitosan concentrations exhibiting the highest CD40 MFI. In terms of CD86 and MHC-II, the 1 mg/mL concentration exhibited higher MFI compared to 2 mg/mL.

### Example 4

### Effect of Chitosan HCl Concentration During NP Preparation

The concentration dependency of chitosan on particle characteristics and immunogenicity was determined, by coating NPs with differing chitosan concentrations and measuring the CD40 and CD86 surface marker expression on the JAWS II cells. The results indicated that the zeta potential exhibits the most significant change in NP characteristics at differing chitosan concentrations, whereas the size remains relatively constant (FIGS. 30A-C). The adsorption efficiency also remains relatively constant at all tested concentrations (FIG. 30D), but the immunogenicity plateaus after 0.5 mg/mL preparatory chitosan concentration (FIGS. 30E-F). This indicates that greater chitosan adsorption does not necessarily correlate to greater immunogenicity.

### Example 5

### Effect of the Incorporation of α-Galactosylceramide (α-GalCer)

The incorporation of α-GalCer, into Chitosan HCl coated ref PLGA NPs, had very little effect on the size and surface charge of the NPs. Quantification of incorporated α-GalCer by LC-MS/MS revealed a high degree of incorporation of the α-GalCer used for preparation (Table 20).

TABLE 20
Particle properties with/without α-GalCer incorporation
(n = 3 independently prepared batches)

| | | | Zeta | α-GalCer |
|---|---|---|---|---|
| | Size | | potential | encapsulation |
| | (nm) | PdI | (mV) | efficiency (%) |
| No α-GalCer | 139.6 ± 2.7 | 0.118 ± 0.025 | 19.9 ± 0.9 | |
| α-GalCer | 137.4 ± 0.2 | 0.148 ± 0.012 | 20.8 ± 1.3 | 97.9 ± 0.6 |

### Example 6

In Vivo Efficacy of PLGA HCl Chitosan NPs with and without α-GalCer

To determine in vivo efficacy, the ref PLGA HCl chitosan NPs with α-GalCer (α-GCCHPSPA-NP) or without (CHPSPA-NP) were tested in a mouse model. Administration into the lungs of the α-GCCHPSPA-NP, resuspended from the spray dried form, resulted in the induction of high serum anti-PspA levels (FIG. 31A). This result also correlated with greater protection against S. pneumoniae, when the vaccinated mice were challenged with ATC6303. The administration of α-GCCHPSPA-NP resulted in greater protection compared to all control groups including the subcutaneously administered PspA (FIG. 31B).

The forgoing embodiments are not intended to limit the scope of the protection afforded by the claims, but rather to describe examples of how the invention may be put into practice.

### REFERENCES

[1] K. Kawakami, N. Yamamoto, Y. Kinjo, K. Miyagi, C. Nakasone, K. Uezu, T. Kinjo, T. Nakayama, M. Taniguchi, A. Saito, Critical role of Vα14+ natural killer T cells in the innate phase of host protection against Streptococcus pneumoniae infection, Eur. J. Immunol., 33 (2003) 3322-3330.
[2] F. Fotouhi, M. Shaffifar, B. Farahmand, S. Shirian, M. Saeidi, A. Tabarraei, A. Gorji, A. Ghaemi, Adjuvant use of the NKT cell agonist alpha-galactosylceramide leads to enhancement of M2-based DNA vaccine immunogenicity and protective immunity against influenza A virus, Arch. Virol., 162 (2017) 1251-1260.
[3] C. Guillonneau, J. D. Mintern, F.-X. Hubert, A. C. Hurt, G. S. Besra, S. Porcelli, I. G. Barr, P. C. Doherty, D. I. Godfrey, S. J. Turner, Combined NKT cell activation and influenza virus vaccination boosts memory CTL generation and protective immunity, Proceedings of the National Academy of Sciences, 106 (2009) 3330-3335.
[4] M. J. Jedrzejas, Pneumococcal virulence factors: structure and function, Microbiol. Mol. Biol. Rev., 65 (2001) 187-207; first page, table of contents.
[5] A. D. Schoofs, Development of a protein antigen based pneumococcal vaccine utilizing a polyanhydride nanoparticle delivery platform, in: Veterinary Microbiology and Preventive Medicine, Iowa State University, 2013.
[6] J. Xu, W. Dai, Z. Wang, B. Chen, Z. Li, X. Fan, Intranasal Vaccination with Chitosan-DNA Nanoparticles Expressing Pneumococcal Surface Antigen A Protects Mice against Nasopharyngeal Colonization by Streptococcus pneumoniae, Clinical and Vaccine Immunology: CVI, 18 (2011) 75-81.
[7] I. G. Kong, A. Sato, Y. Yuki, T. Nochi, H. Takahashi, S. Sawada, M. Mejima, S. Kurokawa, K. Okada, S. Sato, D. E. Briles, J. Kunisawa, Y. Inoue, M. Yamamoto, K. Akiyoshi, H. Kiyono, Nanogel-Based PspA Intranasal Vaccine Prevents Invasive Disease and Nasal Colonization by Streptococcus pneumoniae, Infect. Immun., 81 (2013) 1625-1634.
[8] H. Y. Kang, J. Srinivasan, R. Curtiss, 3rd, Immune responses to recombinant pneumococcal PspA antigen delivered by live attenuated Salmonella enterica serovar typhimurium vaccine, Infect. Immun., 70 (2002) 1739-1749.
[9] D. E. Briles, S. K. Hollingshead, J. C. Paton, E. W. Ades, L. Novak, F. W. van Ginkel, W. H. Benjamin, Jr., Immunizations with pneumococcal surface protein A and pneumolysin are protective against pneumonia in a murine model of pulmonary infection with Streptococcus pneumoniae, J. Infect. Dis., 188 (2003) 339-348.
[10] C. Goulart, T. R. d. Silva, D. Rodriguez, W. R. Politano, L. C. C. Leite, M. Darrieux, Characterization of Protective Immune Responses Induced by Pneumococcal Surface Protein A in Fusion with Pneumolysin Derivatives, PLoS One, 8 (2013) e59605.

1. A nanoparticle comprising a polymer, α-Galactosylceramide (α-GalCer) and chitosan.

2. The nanoparticle as claimed in claim 1, wherein the polymer comprises a synthetic polymer, and optionally, poly(lactic-co-glycolic acid) (PLGA).

3. The nanoparticle as claimed in either claim 1 or 2, wherein the chitosan comprises a water soluble chitosan, and optionally, a chitosan hydrochloride, or a non-animal source chitosan.

4. The nanoparticle as claimed in any preceding claim, wherein the nanoparticle further comprises an antigen.

5. The nanoparticle as claimed in claim 4, wherein the antigen is absorbed on, or conjugated to, a surface of the nanoparticle.

6. The nanoparticle as claimed in claim 5, wherein the antigen is absorbed on to the surface of the nanoparticle by van der waals or electrostatic interaction.

7. The nanoparticle as claimed in any of claims 4 to 6, wherein the antigen is selected from one or more of the following: proteins derived from S. pneumoniae; pneumococcal surface protein A (PspA) and/or pneumolysin (PdT); and derivatives.

8. The nanoparticle as claimed in claim 7, wherein the PspA is recombinant and/or PdT is a detoxified derivative.

9. The nanoparticle as claimed in any preceding claim, in a dry powder format suitable for inhalation.

10. The nanoparticle as claimed in claim 9, wherein the dry powder format is produced by spray drying the nanoparticles.

11. The nanoparticle as claimed in either claim 9 or 10, wherein the nanoparticles are combined with one or more excipients.

12. The nanoparticle as claimed in any one of claims 1 to 8, in a buffered or saline solution or for mixing with a buffered or saline solution.

13. The nanoparticle as claimed in any preceding claim, for initiating an immune response.

14. The nanoparticle as claimed in claim 13, wherein the immune response comprises a systemic and/or local immune response.

15. The nanoparticle as claimed in any preceding claim, for use in the delivery and/or presentation of an antigen.

16. The nanoparticle as claimed in any preceding claim, for use as a medicament.

17. The nanoparticle as claimed in any preceding claim, for use as a medicament formulated for delivery to and/or through mucosa.

18. The nanoparticle as claimed in claim 17, wherein the mucosa is lung mucosa.

19. The nanoparticle as claimed in any one of claims 16 to 18, wherein the medicament is a vaccine.

20. The nanoparticle as claimed in claim 19, wherein the vaccine is a pulmonary vaccine.

21. The nanoparticle as claimed in any one of claims 16 to 18, wherein the medicament is an adjuvant medicament.

22. The nanoparticle as claimed in any preceding claim, for use in the prevention, management, amelioration or treatment of pneumococcal disease or infection.

23. The nanoparticle as claimed in claim 22, wherein the pneumococcal disease comprises pneumonia.

24. The nanoparticle as claimed in any preceding claim, for use as an adjuvant.

25. A multivalent immunogenic composition comprising:
a) a nanoparticle comprising a polymer, α-Galactosylceramide (α-GalCer) and chitosan; and
b) a plurality of capsular polysaccharides from Streptococcus pneumoniae serotypes absorbed on, or conjugated to, a surface of the nanoparticle.

26. The composition as claimed in claim 25, wherein the capsular polysaccharides are selected from one or more of the Streptococcus pneumoniae serotypes: 1, 3, 4, 5, 6A, 6B, 7F, 9V, 14, 18C, 19A, 19F and 23F.

27. The composition as claimed in claim 25 or 26, for use as a medicament.

28. The composition as claimed in claim 27, for use as a medicament formulated for delivery to and/or through mucosa.

29. The composition as claimed in claim 28, wherein the mucosa is lung mucosa.

30. The composition as claimed in any one of claims 27 to 29, wherein the medicament is a vaccine.

31. The composition as claimed in claim 30, wherein the vaccine is a pulmonary vaccine.

32. The composition as claimed in any one of claims 27 to 31, wherein the medicament is an adjuvant medicament.

33. The composition as claimed in any preceding claim, for use in the prevention, management, amelioration or treatment of pneumococcal disease or infection.

34. The composition as claimed in claim 33, wherein the pneumococcal disease comprises pneumonia.

35. A method of producing a nanoparticle for the delivery and/or presentation of one or more antigens, the method comprising:
a) forming an oil-in-water emulsion of a polymer and α-Galactosylceramide (α-GalCer) in an organic solvent and agitating the emulsion;
b) mixing the emulsion with an aqueous phase containing chitosan so as to form a mixture and removing the organic solvent from the mixture so as to form a nanoparticle suspension;
c) removing any unbound α-Galactosylceramide (α-GalCer) and/or chitosan from the nanoparticle suspension;
d) absorbing or conjugating the one or more antigens on to the nanoparticles; and optionally separating the nanoparticles which have absorbed or conjugated antigens present.

36. The method as claimed in claim 35, wherein the method further comprises:
e) drying the nanoparticles.

37. The method as claimed in claim 36, wherein e) comprises spray drying, and optionally comprises spray drying the nanoparticles together with one or more excipients.

38. The method as claimed in any one of claims 35 to 37, wherein the oil-in water emulsion in a) further comprises polyvinyl alcohol (PVA), and optionally, the PVA is removed in b).

39. The method as claimed in to any one of claims 35 to 38, wherein the agitation in a) is by sonification.

40. The method as claimed in any one of claims 35 to 39, wherein the nanoparticles are formed having a diameter in the range of 100 to 500 nm.

41. The method as claimed in any one of claims 36 to 39, wherein the nanoparticles are embedded within microcarrier formed by spray drying and form particles having a diameter in the range of 1 to 5 μm.

42. The method as claimed in any one of claims 35 to 41, wherein the polymer comprises poly(lactic-co-glycolic acid) (PLGA).

43. The method as claimed in any one of claims 35 to 42, wherein the chitosan comprises chitosan hydrochloride or a non-animal source chitosan.

44. The method as claimed in any one of claims 35 to 43, wherein the one or more antigens are absorbed on to the surface of the nanoparticle by van der waals or electrostatic interaction.

45. The method as claimed in any one of claims 35 to 44, wherein the one or more antigens are selected from one or more of the following: pneumococcal surface protein A (PspA) and/or pneumolysin (PdT) and/or derivatives thereof.

46. The method as claims in claim 45, wherein the PspA is recombinant and/or PdT is a detoxified derivative.

47. The method as claimed in any one of claims 35 to 44, wherein the one or more antigens comprise a plurality of capsular polysaccharides from Streptococcus pneumoniae serotypes.

48. The method as claims in claim 47, wherein the capsular polysaccharides are selected from one or more of the Streptococcus pneumoniae serotypes: 1, 3, 4, 5, 6A, 6B, 7F, 9V, 14, 18C, 19A, 19F and 23F.

49. The method as claimed in anyone of claims 35 to 48 for the production of a nanoparticle as claimed in anyone of claims 1 to 24 or a multivalent immunogenic composition as claims in any one of claims 25 to 34.
